(19) United States
(12) Patent Application Publication
(10) Pub. No.: US 2014/0194683 A1
(43) Pub. Date: Jul. 10, 2014

(54) TROCAR, AND SURGERY ASSISTANCE SYSTEM

(71) Applicants: National University Corporation Chiba University, Chiba-shi (JP); Advanced Healthcare Co., Ltd., Tokyo (JP)

(72) Inventor: Toshiya NAKAGUCHI, Funabashi-shi (JP)

(73) Assignees: National University Corporation Chiba University, Chiba-shi (JP); Advanced Healthcare Co., Ltd., Tokyo (JP)

(21) Appl. No.: 13/974,889

(22) Filed: Aug. 23, 2013

(30) Foreign Application Priority Data
Jan. 10, 2013 (JP) 2013-002337

(51) Int. Cl. A61B 17/34; A61B 17/29

(52) U.S. Cl. CPC A61B 17/3415; A61B 17/29
USPC 600/109

(57) ABSTRACT

Problem:

To provide a trocar equipped with a retractable camera, with which the security is enhanced as compared with the related art.

Means for Solution:

An opening portion 13 is provided at a position of a pipe portion 11. The position of opening portion 13 is reliably within the body of the patient. A shaft 14 is arranged along the interior of the trocar, along an edge of the opening portion 13. Several bearings 15 are fixed to the inter wall of the pipe portion 11, with the shaft 14 being rotatably mounted in the bearings 15. The end portion of the shaft 14 extends to the exterior of the trocar, outside the body of the patient. A selection lever 16 is provided at the outer end portion of the shaft 14. The selection lever 16 can be changed over between a stored position and a deployed position, and can be fixed in each of these positions. A camera 17 is rigidly and integrally attached to the shaft 14 at a position that corresponds to the opening portion 13. A cable 18 is connected to the camera 17, extends through the interior of the trocar 1, and is connected to an external image processing device 6.

### TECHNICAL FIELD

The present invention relates to a trocar and to a surgery assistance system that includes a trocar, and in particular relates to a trocar that is equipped with a retractable camera.

### BACKGROUND ART

In recent years, in order to maintain and enhance the QOL (quality of life) of patients, surgical operations of low invasiveness are being performed using laparoscopic surgery and so on. In abdominal laparoscopic surgery, carbonic acid gas is injected into the abdominal cavity so that the abdominal wall is distended, and thereby space and a good field of view for manipulation are ensured. A small hole is formed in the abdominal wall, and an instrument called a trocar is inserted. Then, usually, a laparoscope (i.e. a CCD camera) and a forceps (which is a surgical instrument) are inserted into the interior of the body of the patient, and the required surgical operation is performed while observing an image displayed upon a monitor by the laparoscope.

Now this operation is performed with a narrow field of view, since reliance is placed only upon the image obtained from the laparoscope, and this imposes a burden upon the surgeon. But if another hole is made in the abdominal wall for inserting another camera in order to widen the field of view, then the advantage of minimum invasiveness is lost.

Thus, the present inventors have noticed that the trocar always remains fixed in the abdominal wall, and have proposed a trocar that includes a mechanism for storing and deploying a retractable camera (refer to Non Patent Document #1). By using a forceps trocar in this manner, no new hole need be opened in the abdominal wall, and the advantage of minimum invasiveness is not lost. With this system in which the image obtained from the retractable camera is used in addition to the image obtained from the laparoscope. it is possible to eliminate narrowing of the field of view.

### CITATION LIST

### Non-Patent Literature

Non-Patent Document #1: Takuya Okubo, Toshiya Nakaguchi, et al., “Laparoscopic Surgery Assistance System by using Retractable Camera”, Proceedings of Japanese BioMedical Engineering Symposium 2011, CD ROM Abstracts, Presentation No. 252.

### SUMMARY OF THE INVENTION

### Technical Problem

The structure of a trocar having a retractable camera according to the related art will now be explained. The trocar includes a pipe portion and a head portion. An opening portion is provided at a position on the pipe portion. The position of the opening portion is definitely inside the body of the patient when the trocar is inserted. A hinge mechanism that is rotatable (pivotable) along the axial direction of the pipe portion is provided at one edge portion of this opening portion, and a camera is connected to the pipe portion via this hinge mechanism. A torsion spring is provided to the hinge mechanism, and normally the elastic force of the torsion spring operates to deploy the camera out through the opening portion. Furthermore, a cable that extends to the outside is linked to the camera, and, when this cable is pulled, the camera is moved into the opening portion against the resistance of the elastic force of the torsion spring which is overcome, and is stored in the opening portion. In other words, the camera can be changed over between the stored position and the deployed position in this manner.

When the pipe portion is to be inserted into a hole in the abdominal wall of the patient, the cable is pulled and the camera is held in the stored position, and after the pipe portion has been inserted, the cable tension is released, and the camera goes into the deployed position. Photography is performed in this state, and, when the pipe portion is to be withdrawn after the operation, the cable is again pulled and the camera goes back into the stored position.

The present inventors have manufactured a trial trocar of this type that is provided with a retractable camera, and have confirmed its usefulness by performing animal experiments and demonstration experiments using a mannequin. However, when opinions from surgeons who perform operations have been obtained, it has been commented that there may be a danger in relation to security. Namely, there may be a danger of cutting the cable when inserting or withdrawing an operating instrument (for example, a forceps) through the trocar. As a result, it may become impossible to store the camera away, so that it may become difficult to extract the trocar from the body of the patient. In the worst case, it may be necessary to cut open the abdominal cavity to get the trocar out, and this not only sacrifices the advantage of minimum invasiveness of the body of the patient, but also poses a significant risk to the patient.

The present invention has been conceived in order to solve the problem described above, and its object is, while maintaining functionality equivalent to that in the related art, to provide a trocar incorporating a retractable camera, and a surgery assistance system including such a trocar, with which security is enhanced.

### Means for Solution

In order to solve the problem described above, the present invention proposes a trocar for being passed through an abdominal wall of a patient, comprising: a pipe portion that inserts a surgical instrument into the interior of the body of the patient; an opening portion that is provided at a position of said pipe portion that, during surgery, is within the body of the patient; a shaft that is rotatably (pivotably) mounted in said pipe portion and that extends in the axial direction of said pipe portion, along an edge of said opening portion thereof, one end portion of said shaft being external to the trocar; and a camera that is rigidly attached to said shaft; and wherein, by said end portion of said shaft being rotated, said camera is changed over between a stored position in which it is stored within the trocar and a deployed position in which it is deployed to the exterior of the trocar and is capable of photography.

More desirably, said surgical instrument is a forceps.

Since, with a trocar equipped with a retractable camera according to the related art, the camera is deployed by the elastic force of a torsion spring and is stored by a cable being pulled, accordingly there may be a danger that, if the cable should be disconnected by a forceps, it might become impossible to store the camera.

However, with the trocar equipped with a retractable camera according to the present invention, the camera is changed over between the stored position and the deployed position by rotation of the shaft, and accordingly there is no danger of any cable becoming disconnected. Moreover, the camera and the shaft are rigidly joined together, so that the durability is high. Furthermore, there is almost no danger of failure, because the structure is simple. As a result, the security is enhanced.

And, in order to solve the problem described above, the present invention proposes a surgery assistance system, including: a laparoscope; a forceps trocar having a retractable camera that, by rotation of a shaft, can be changed over between a stored position and a deployed position; and an image processing device that performs processing to combine an image obtained from said laparoscope and an image obtained from said retractable camera.

Due to this, it is possible to widen the field of view in safety.

Moreover, in order to solve the problem described above, the present invention proposes a surgery assistance system, including: a plurality of forceps trocars, each having a retractable camera that, by rotation of a shaft, can be changed over between a stored position and a deployed position; and an image processing device that performs processing to combine images obtained from said retractable cameras.

Due to this, it is possible to widen the field of view in safety. Moreover the invasiveness is reduced, since no laparoscope is required.

More desirably, this surgery assistance system further includes a projector that is provided above the operating table, and that projects the combined image onto the abdomen of the patient.

With this arrangement, the line of sight of the surgeon and the direction of his field of operation coincide with one another, so that he can really experience the same feeling as that during an open-abdomen operation.

Yet further, in order to solve the problem described above, the present invention proposes a port for being passed through a chest wall of a patient, comprising: a pipe portion that inserts a surgical instrument into the interior of the chest of the patient; an opening portion that is provided at a position of said pipe portion that, during surgery, is within the body of the patient; a shaft that is rotatably (pivotably) mounted in said pipe portion and that extends in the axial direction of said pipe portion, along an edge of said opening portion thereof, one end portion of said shaft being external to the port; and a camera that is rigidly attached to said shaft; and wherein, by said end portion of said shaft being rotated, said camera is changed over between a stored position in which it is stored within the port and a deployed position in which it is deployed to the exterior of the port and is capable of photography.

### Advantageous Effect of the Invention

According to the present invention, it is possible to enhance the security, as compared to a trocar according to the related art.

### BRIEF DESCRIPTION OF THE DRAWINGS

FIG. 1A is a perspective view of a trocar having a retractable camera;

FIG. 1B is a perspective view of this trocar having a retractable camera, from another point of view;

FIG. 2 is a sectional view of this trocar;

FIG. 3A is a figure showing the state of this trocar with the camera in the stored position;

FIG. 3B is a figure showing the state of this trocar with the camera in the deployed position;

FIG. 4A is a perspective view of a trocar according to the related art (with the camera in the deployed position);

FIG. 4B is a perspective view of this trocar according to the related art (with the camera in the stored position);

FIG. 5 is a figure showing a surgery assistance system according to a first embodiment;

FIG. 6 is a figure showing a surgery assistance system according to a variant embodiment;

FIG. 7 is a figure showing a surgery assistance system according to a second embodiment; and

FIG. 8 is a figure showing a surgery assistance system according to a third embodiment.

### DESCRIPTION OF THE EMBODIMENTS

### A Trocar Equipped with a Retractable Camera

### (Structure)

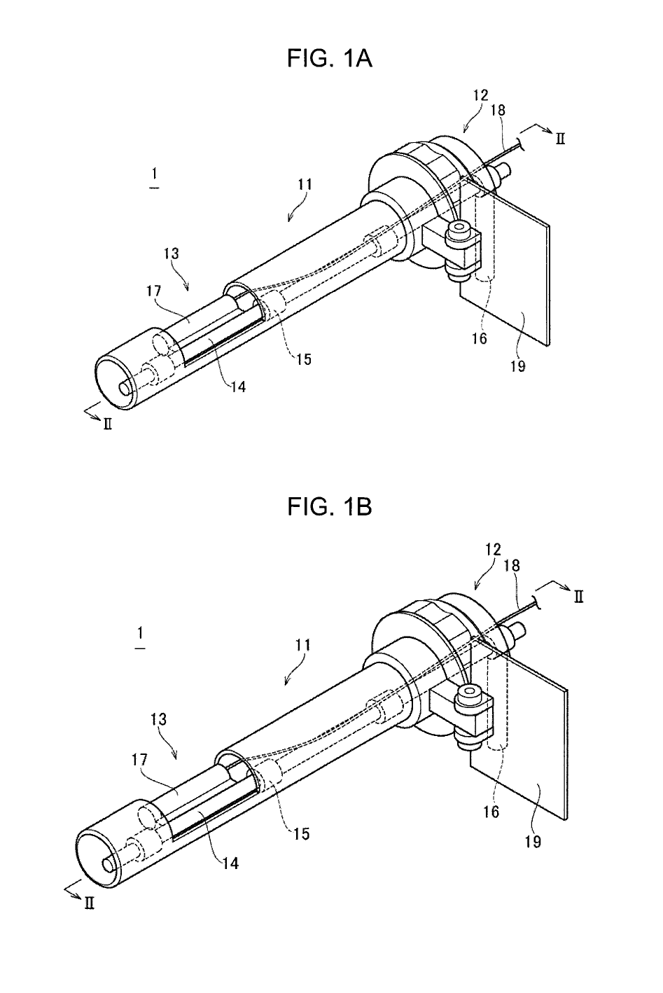

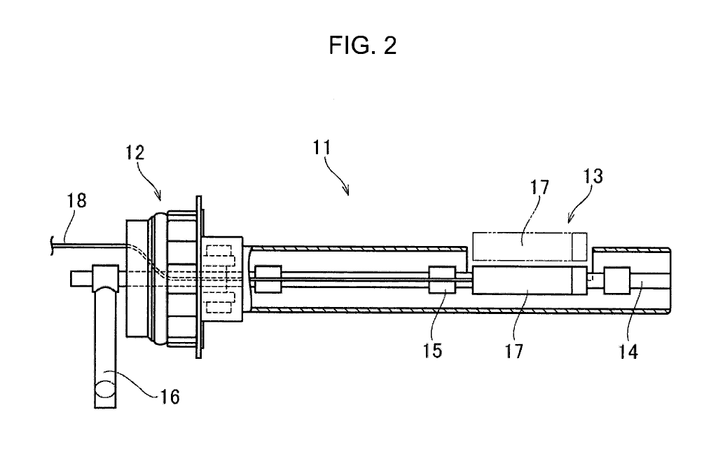

The structure of a trocar equipped with a retractable camera will now be described. FIG. 1 shows two perspective views of a trocar 1 according to this embodiment that is equipped with a retractable camera. FIG. 1A and FIG. 1B show this trocar from different points of view. And FIG. 2 is a sectional view.

The trocar 1 comprises a pipe portion 11 and a head portion 12. The greater part of the pipe portion 11 is inserted into a hole in the abdominal wall of the patient. The head portion 12 is provided as connected to the upper portion of the pipe portion 12. The head portion 12 is hollow, and a forceps can be inserted thereinto from above. Moreover, although the details thereof are not shown, the head portion 12 is provided with a sealing mechanism that prevents air leakage when the forceps is inserted and withdrawn, and with an air blowing mechanism that injects air into the abdominal cavity.

The specific distinguishing feature of this embodiment will now be explained. An opening portion 13 is provided at a position of the pipe portion 11. The position of the opening portion 13 can be relied upon to be within the body of the patient when the trocar has been inserted. A shaft 14 is disposed along the axial direction of the pipe portion, and extends along one edge of the opening portion 13. A plurality of bearings 15 are fixed to the inner wall of the pipe portion 11, and these bearings 15 hold the shaft 14 so that it can be rotated (pivoted). The end portion of the shaft 14 projects to the exterior of the trocar. A selection lever 16 is fixed to the end portion of the shaft 14. This selection lever 16 can be rotated between a stored position and a deployed position, and can be retained in each of these positions. For example, a detent mechanism not shown in the figures may be used to retain.

A camera 17 is rigidly and integrally fixed to the shaft 14 at a position that corresponds to the opening portion 13. A cable 18 is connected to the camera 17, and this cable 18 is led out through the trocar 1 and is connected to an external image processing device 6 (to be described hereinafter).

It should be understood that while, in the shown configuration, the cable 18 is led out along the shaft 18, it would be even more desirable to make the shaft 14 hollow, and to lead out the cable 18 through the shaft 14; in this case, there would be no danger of the cable 18 becoming disconnected during insertion of the forceps.

### (Operation)

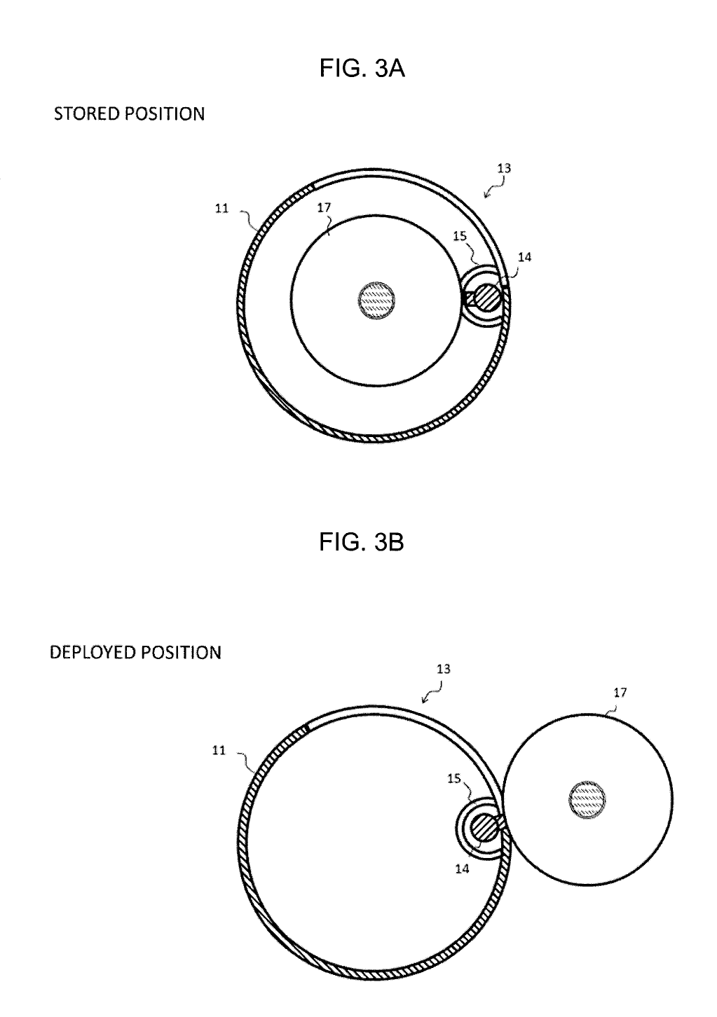

The operation of this trocar equipped with a retractable camera will now be explained. FIG. 3A is a figure showing the state with the camera 17 in the stored position, while FIG. 3B is a figure showing the state with the camera 17 in the deployed position. Both of these figures are sectional views of the pipe portion 11 at a position corresponding to the opening portion 13.

By the selection lever 16 being rotated (in other words, by rotation of the shaft 14), the camera 17 may be changed over between the stored position in which it is stored within the trocar and the deployed position in which it is deployed to the exterior of the trocar for photography to be performed.

When the pipe portion 11 is to be inserted into a hole in the abdominal wall of the patient, the selection lever 16 is fixed in the stored position, and holds the camera 17 in the stored position via the shaft 14 (refer to FIG. 3A). Due to this, it is possible to insert the pipe portion 11 through the hole in the abdominal wall without the camera 17 causing any hindrance. After the pipe portion 11 has been inserted, the selection lever 16 is moved over to the deployed position and is fixed there, so that the camera 17 is moved to the deployed position via the shaft 14 (refer to FIG. 3B). Photography is performed in this state, and then, when the pipe portion 11 is to be withdrawn after the operation has been completed, the selection lever 16 is moved back to the stored position and fixed there, so that the camera 17 is moved back to the stored position via the shaft 14 (refer to FIG. 3A). Due to this, it is possible to withdraw the pipe portion 11 from the hole in the abdominal wall without any hindrance being caused by the camera 17.

### (Beneficial Effects)

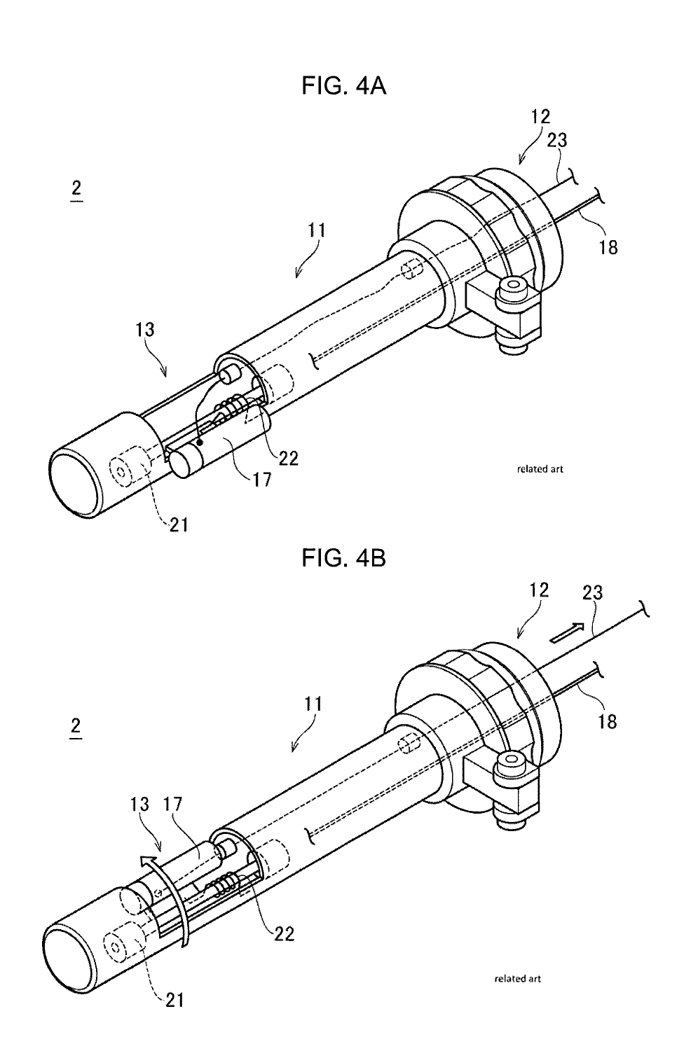

The beneficial effects of this embodiment will now be explained by comparison with the related art. FIG. 4 shows two perspective views of a trocar 2 according to the related art. FIG. 4A is a figure showing this related art trocar 2 in the deployed state with the camera 17 in the deployed position, while FIG. 4B is a figure showing this related art trocar 2 in the stored state with the camera 17 in the stored position. The same components of this embodiment are denoted by the same reference symbols. The trocar 2 comprises a pipe portion 11 and a head portion 12. An opening portion 13 is provided at a position of the pipe portion 11 that is inserted into the body of the patient. A rotatable hinge mechanism 21 is provided along an edge of the opening portion 13 of the pipe portion 11. The edge extends along the axial direction. A camera 17 is connected to the pipe portion 11 via this hinge mechanism 21. A torsion spring 22 is provided to the hinge mechanism 21, and normally the elastic force of this torsion spring 22 acts to deploy the camera 17. On the other hand, a tension cable 23 is connected to the camera 17 and extends to the exterior of the trocar, and, when this tension cable 23 is pulled, the camera 17 is stored in the opening portion 13 against the resistance of the elastic force of the torsion spring 22 which is overcome. And a cable 18 is connected to the camera 17.

When the pipe portion 11 is to be inserted into a hole in the abdominal wall of the patient, the tension cable 23 is pulled and the camera 17 is held in the stored position, and, after the pipe portion 11 has been inserted, the tension in the tension cable 23 is slackened, and the camera 17 moves over to the deployed position. Photography is performed in this state, and then, when the pipe portion 11 is to be withdrawn after the operation has been completed, the tension cable 23 is again pulled, so that the camera 17 is moved back to the stored position.

However there may be a danger that, when a forceps is inserted into the trocar 2 or is pulled out, the tension cable 23 may be disconnected or cut. In this case, due to the elastic force of the torsion spring 22, the camera 17 will remain in the deployed state, and thus it will constitute an obstruction that makes it difficult to extract the trocar 2 from the body of the patient.

By contrast, with this embodiment, the camera 17 is changed over between the stored position and the deployed position by rotation of the selection lever 16 (i.e. by rotation of the shaft 14). In other words, there is no danger of disconnection or cutting of any tension cable, as in the case of the related art. The selection mechanism of this invention has a simple structure so that there is almost no danger of it going wrong, and moreover it is durable due to the firm connection between the camera 17 and the shaft 14, so that the security level is enhanced.

### A Surgery Assistance System

A that uses a trocar equipped with a retractable camera will now be explained. Embodiments #1 through #3 of this surgery assistance system are shown below. While the beneficial effects due to the various characteristic features of the present invention will be described below, it should be appreciated that it is possible for the surgeon to take advantage of his current fund of knowledge and experience relating to conventional operational technique, since each of these embodiments is based upon conventional prior art abdominal laparoscopic surgery, and since there is no very great change in the operational method.

Moreover, the way in which the improved trocar (above embodiment) is employed is simple, and it is possible still to apply a surgery assistance system of an already existing type with a few simple improvements.

### The First Embodiment

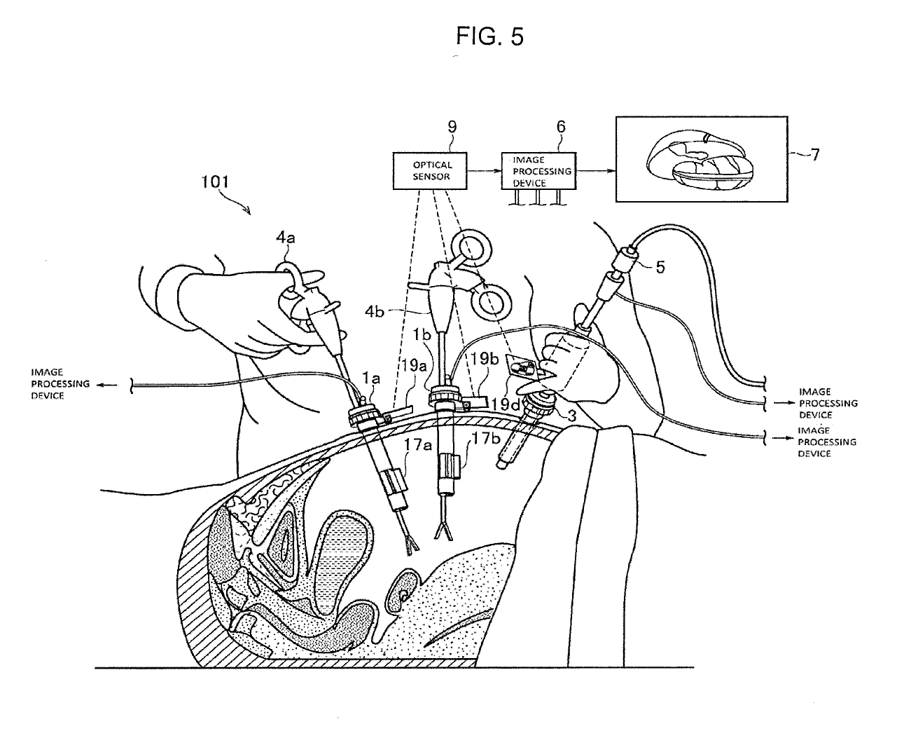

FIG. 5 is a figure showing the general structure of a surgery assistance system 101. The surgery assistance system 101 comprises forceps trocars 1a and 1b that are respectively equipped with retractable cameras 17a and 17b, a laparoscope trocar 3, forceps 4a and 4b, a laparoscope 5, an image processing device 6 that inputs images obtained from the retractable cameras 17a and 17b and an image obtained from the laparoscope 5 and performs processing to combine these images, and a monitor 7 that outputs the combined image resulting from this combination processing performed by the image processing device 6.

The forceps 4a and 4b are one type of surgical instrument, and are used for grasping, holding down, pulling, and cutting blood vessels and organs and so on. Each of them is generally formed as a pair of scissors, and its inner end portion is operated by outer gripping portions being rotated around a fulcrum. When the gripping portions are closed together, these forceps can be inserted through the trocar 1a, 1b. It should be understood that while, generally, a plurality of forceps are used in abdominal laparoscopic surgery, at least one forceps and one forceps trocar are enough for application of this system.

The laparoscope 5 is one type of endoscopic instrument, and comprises a camera and a light source. The laparoscope 5 is inserted into the body of the patient by being passed through the laparoscope trocar 3.

Now, in conventional abdominal laparoscopic surgery according to the prior art, the field of view is narrow, since only the image obtained from the laparoscope is relied upon. If a new hole is opened in the abdominal wall in order to insert another camera for enlarging the field of view, then the advantage of low invasiveness is lost.

By contrast, in this embodiment, it is possible to insert a plurality of cameras into the abdominal cavity by using the trocars 1a and 1b that are equipped with the retractable cameras 17a and 17b. Due to this, it is possible to enlarge the field of view. Moreover, since the retractable cameras 17a and 17b reliably photograph the end portions of the forceps 4a and 4b, accordingly it is possible reliably to obtain images of the actual spots where cutting or the like is being performed, which are very important images.

Furthermore, due to the use of the forceps trocars for the cameras as well, it is not necessary to open any new hole in the abdominal wall, so that the advantage of low invasiveness is maintained.

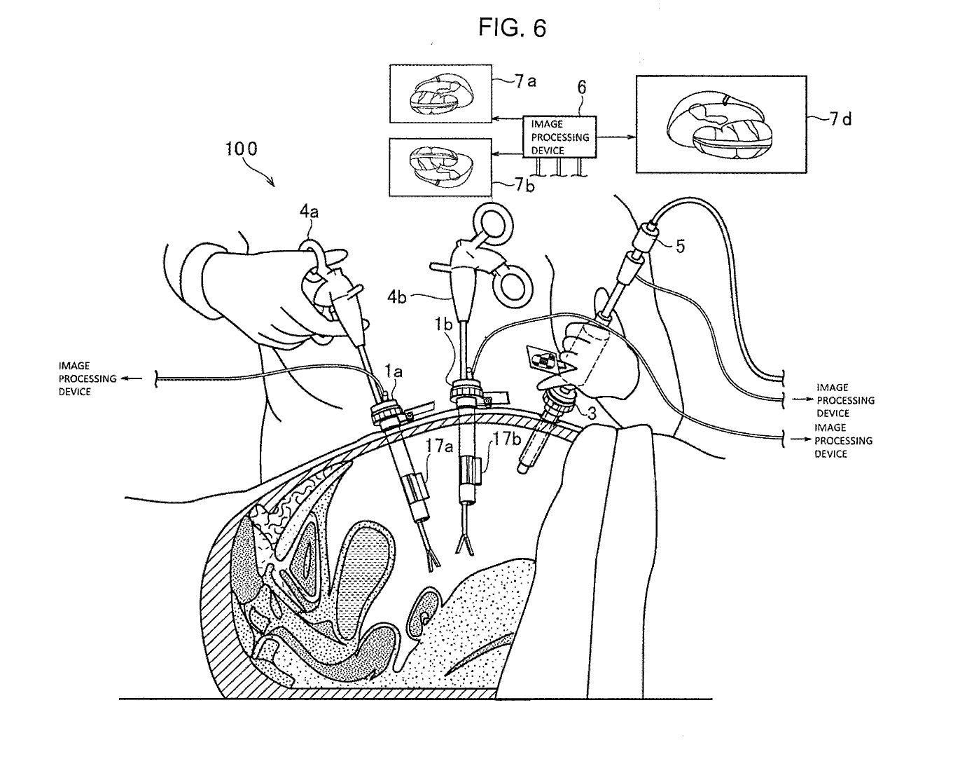

It would also be acceptable to arrange to output the images obtained from the retractable cameras 17a and 17b of the trocars 1a and 1b and the image obtained from the laparoscope 5 to respective separate monitors (for this variant embodiment; refer to FIG. 6). However, if the operation is performed while the surgeon is looking at a plurality of monitors, there may be a danger of him becoming distracted and losing his concentration.

In this embodiment, the image processing device 6 performs combination processing for combining the plurality of images, and the resulting combined image is outputted upon the monitor 7. The surgeon is able to obtain a wide field of view by looking at the monitor 7. Due to this, the burden upon the surgeon is alleviated.

### The Second Embodiment

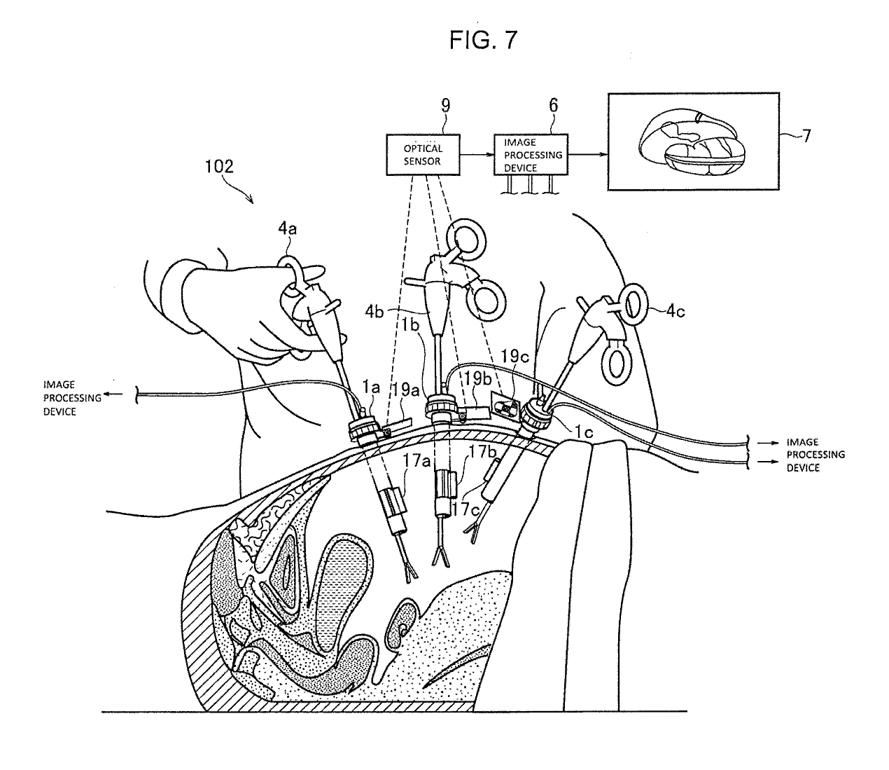

FIG. 7 is a figure showing the general structure of a surgery assistance system 102. This surgery assistance system 102 comprises forceps trocars 1a, 1b, and 1c that are respectively equipped with retractable cameras 17a, 17b, and 17c, forceps 4a, 4b, and 4c, an image processing device 6 that inputs images obtained from the retractable cameras 17a, 17b, and 17c and performs processing to combine these images, and a monitor 7 that outputs the combined image resulting from this combination processing by the image processing device 6.

In other words, the laparoscope trocar 3 and the laparoscope 5 in the surgery assistance system 101 of the first embodiment are omitted, and another forceps trocar 1c including a retractable camera 17c and another forceps 4c are added instead.

It should be understood that while, generally, a plurality of forceps are used in abdominal laparoscopic surgery, at least two forceps and two forceps trocars are enough for the application of this system according to the second embodiment. Moreover it should be understood that, although no laparoscope is actually used in this embodiment, for convenience it is referred to as laparoscopic surgery.

By contrast to the situation when a laparoscope 5 is used as in the first embodiment, in which the surgeon needs actively to orient the laparoscope 5 in order to take a photograph of the spot where cutting or the like is being performed, in this embodiment, since the retractable camera 17 reliably photographs the end portion of the forceps 4a, accordingly it is possible reliably to obtain an image of the actual spot where cutting or the like is being performed, which is a very important image. Thus, on the supposition that that the performance of the retractable camera 17 is high, it is possible to obtain an image of higher quality than that obtained with a laparoscope.

Furthermore, by dispensing with the laparoscope trocar 3 and the laparoscope 5, it becomes unnecessary to make any hole in the abdominal wall for passing these, so that the invasiveness becomes yet lower.

However, instead of the light source that is provided to the laparoscope 5, it is necessary to provide light sources to the trocars 1 (or to the cameras 17).

### The Third Embodiment

The third embodiment is a variant of the first and second embodiments. While in the first and second embodiments the surgeon performs an operation by manipulating the forceps 4 and the laparoscope 5 while looking at the monitor 7, there is a discrepancy between the line of sight of the surgeon and the direction towards the actual field of operation, so that the surgeon experiences a sense of discomfort, and this constitutes a burden. In particular, a surgeon who has performed a lot of open-abdomen operations sometimes finds it difficult to get used to abdominal laparoscopic surgery.

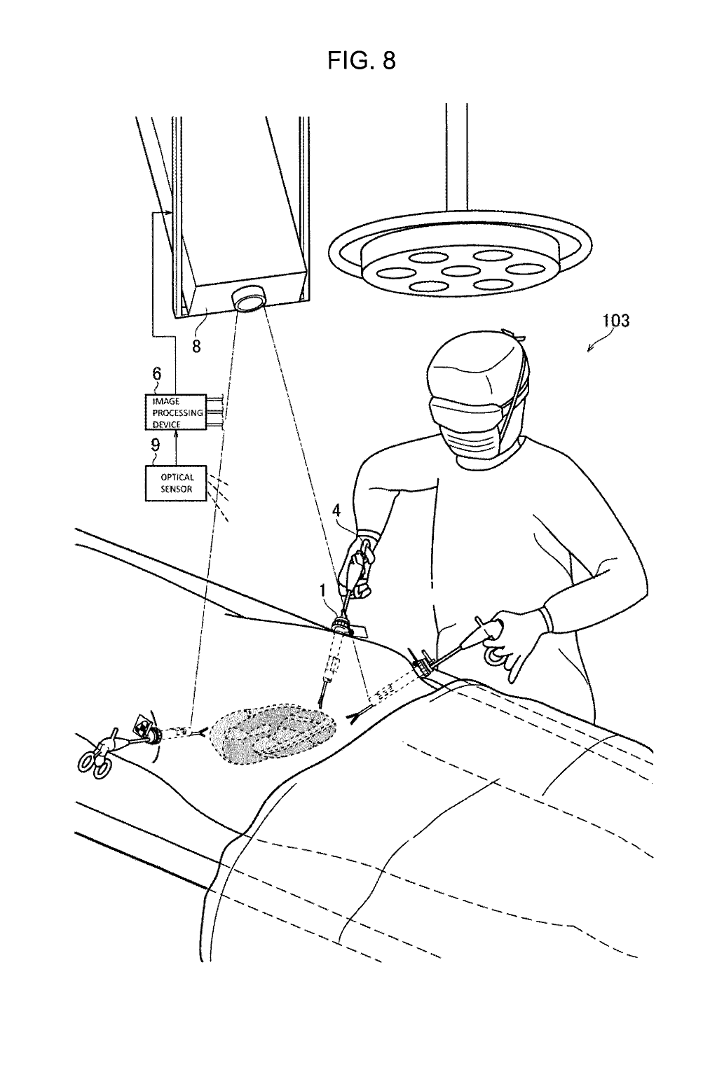

FIG. 8 is a figure showing the general structure of a surgery assistance system 103. Elements that are the same as ones in the first and second embodiments are omitted as appropriate. This surgery assistance system 103 comprises a projector 8, instead of the monitor 7. The projector 8 is provided over the operating table, and projects the combined image resulting from combination processing by the image processing device 6 directly upon the abdominal portion of the patient.

Due to this, the line of sight of the surgeon and the direction of his field of operation coincide, so that he is able to experience the same feeling of reality as during an open-abdomen operation. This means that the burden upon the surgeon is alleviated.

### Port Having Retractable Camera

While the above explanation has been expressed in terms of abdominal laparoscopic surgery, the present invention can also be applied to chest laparoscopic surgery. However, the operating instrument that is termed a “trocar” in abdominal laparoscopic surgery is called a “port” in chest laparoscopic surgery. That is to say, a trocar and a port are devices of almost the same type.

### EXPLANATION OF THE REFERENCE SYMBOLS

1: trocar

2: trocar (related art)

3: trocar (for laparoscope)

4: forceps

5: laparoscope

6: image processing device

7: monitor

8: projector

9: optical sensor

11: pipe portion

12: head portion

13: opening portion

14: shaft

15: bearing

16: selection lever

17: camera

18: cable

19: marker

21: hinge mechanism

22: torsion spring

23: tension cable

101-103: surgery assistance systems

1. A trocar comprising:
a pipe portion that inserts a surgical instrument into an interior of a body of a patient;
an opening portion that is at a position of the pipe portion that, during surgery, is within the body of the patient;
a shaft that is rotatably mounted in the pipe portion and extends in an axial direction of the pipe portion, along an edge of the opening portion thereof, wherein one end portion of the shaft is external to the trocar; and
a camera that is rigidly attached to the shaft;
wherein
when the end portion of the shaft is rotated, the camera is changed over between a stored position in which it is stored within the trocar and a deployed position in which it is deployed to an exterior of the trocar and is capable of photography.

2. The trocar according to claim 1, wherein the surgical instrument is a forcep.

3. A surgery assistance system, comprising:
a trocar according to claim 1;
a laparoscope; and
an image processing device that performs processing to combine an image obtained from the laparoscope and an image obtained from the camera, which is retractable.

4. A surgery assistance system, comprising:
a plurality of trocars according to claim 1; and
an image processing device that performs processing to combine images obtained from a plurality of the cameras, which are retractable.

5. The surgery assistance system according to claim 3, further comprising a projector above the operating table, which projects a combined image onto an abdomen of the patient.

6. A port comprising:
a pipe portion that inserts a surgical instrument into an interior of a chest of a patient;
an opening portion that is at a position of the pipe portion that, during surgery, is within a body of the patient;
a shaft that is rotatably mounted in the pipe portion and extends in an axial direction of the pipe portion, along an edge of the opening portion thereof, wherein one end portion of the shaft is external to the port; and
a camera that is rigidly attached to the shaft;
wherein
when the end portion of the shaft is rotated, the camera is changed over between a stored position in which it is stored within the port and a deployed position in which it is deployed to an exterior of the port and is capable of photography.
